(19) United States
(12) Patent Application Publication
(10) Pub. No.: US 2002/0192175 A1
(43) Pub. Date: Dec. 19, 2002

(54) Hair relaxer and coloring system

(76) Inventors: Manilal Patel, Bensonville, IL (US); Melissa Nava, Chicago, IL (US)

Correspondence Address: BREZINA & EHRLICH, Suite 333, 47 West Polk Street, Chicago, IL, 60605-2092, US

(21) Appl. No.: 09/837,405

(22) Filed: Apr. 18, 2001

(51) Int. Cl. A61K 7/09; A61K 7/13

(52) U.S. Cl. 424/70.2; 8/426

(57) ABSTRACT

A composition for straightening and relaxing hair, contains, by weight, about 0.1-10% of at least one cationic dye, about 1-7% of an alkali relaxer, about 7-25% of an emulsifier, about 10-32% of an emollient, about 5-15% of a moisturizer, about 0.5-3% of a conditioning agent and about 35-55% of water.

### BACKGROUND OF THE INVENTION

1. Field of the Invention

The invention relates to the field of hair treatment products, and specifically to a single product which can both relax the hair and dye the hair.

2. Description of Related Art

A combination hair dye and relaxer system would be desirable, saving the consumer a step in hair treatment. Many dyes, however, are not compatible with the harsh chemicals, typically alkalis, used in relaxer systems.

Recently, Warner Jenkinson Europe has suggested that its Arianor® dyes can be used in a hair relaxer system. The system disclosed in the Arianor Hair Colour System Formulation Index contains, by weight, 2% sodium hydroxide, 20% liquid paraffin, 5% emulsifier, 3% thickener, 1% dye and water to 100%. This system, however, is unsatisfactory from the standpoint of the consumer, leaving the hair unmanageable and the scalp irritated and damaged.

### SUMMARY OF THE INVENTION

It is therefore an object of the invention to provide a single composition that can provide the diverse functions of satisfactory coloring of hair and simultaneously relaxing and straightening the hair, while also conditioning and softening the hair.

It is a further object of the invention to provide a single composition for relaxing and straightening which can be provided in a single package with extended shelf life, or in a two package formulation for mixing before use.

To achieve these and other objects, the invention is directed to a composition for coloring and relaxing hair, comprising, by weight, 0.05 to 10% of a cationic dye, 15-45% by weight of at least one oily material selected from the group consisting of mineral oil and petrolatum, 1 to 5% of at least one alkali selected from the group consisting of sodium hydroxide, potassium hydroxide, lithium hydroxide and calcium hydroxide, 1 to 20% of at least one conditioning agent, remainder water and adjuvants constituting a cosmetically acceptable medium.

### DETAILED DESCRIPTION OF THE INVENTION

The dyes which are useful in the practice of the invention are cationic, diazo dyes of the general formulae:

(W—NH—Z−X⁺) Y⁻ (I)

or

(W—N=N—Z−X⁺) Y⁻ (II)

where Z is a single benzene ring or a naphthalene ring, W is a substituted or unsubstituted ring with 5 or 6 members, or a naphthalene ring. X⁺is a quaternary ammonium group of typical formula (R₁R₂R₃)N⁺ where R₁, R₂, and R₃ are, independently, short chain alkyl groups with 1-4 carbons, and are preferably methyl groups. Y⁻ is an anion, preferably Cl⁻.

Cationic, diazo dyes are well known, disclosed, for example, in U.S. Pat. Nos. 5,980,587 and 5,879,413, and in WO95/14144 and WO95/01772, which are incorporated herein by reference. A particularly preferred group of dyes is sold under the name Arianor® by Warner Jenkinson Europe. Useful Arianor® dyes include the following:

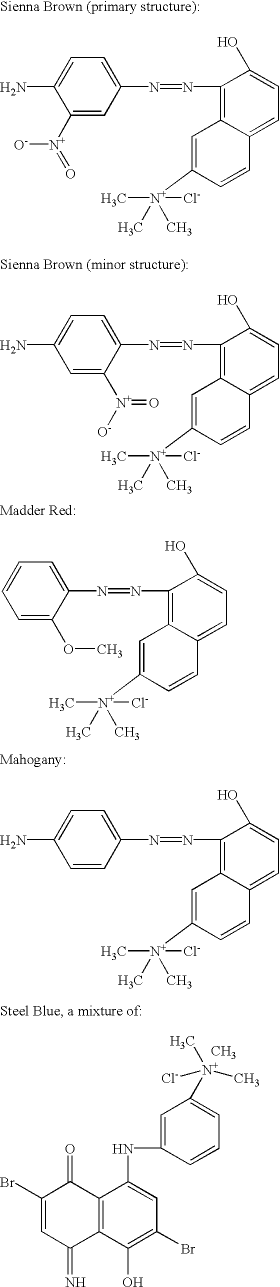

While the above Arianor® dyes are preferred, other cationic dyes may also be used, such as those disclosed in U.S. Pat. Nos. 5,879,413 and 5,980,587.

The compositions of the invention also contain an effective straightening formula which does not damage the hair. The straightening formula is typically based on an alkali, for example, sodium hydroxide, lithium hydroxide or calcium hydroxide, with an oily carrier, for example, mineral oil or petrolatum, and an emulsifier. However, compositions so limited tend to damage the hair, and therefore, conditioning agents are included.

The preferred conditioning agent is polymethacrylamido-propyltrimonium chloride, disclosed in U.S. Pat. No. 5,476,650. The conditioning agent is used in conjunction with petrolatum as a moisturizer, mineral oil and PEG-75 lanolin as emollients, and DEA-Oleth-10 phosphate, cetyl alcohol, ceteareth-20 and cetearyl alcohol as emulsifiers. The conditioning agent mitigates hair damage by helping to facilitate the penetration of the active ingredients into the hair shaft during the relaxing to allow a more efficient relaxing process. When the relaxer contains guanidine hydroxide, the polymethacryl-amidopropyltrimonium chloride, works synergistically with the guanidine hydroxide to provide faster and more thorough relaxing.

The dye-relaxer formulations of the invention have the advantage that they can be formulated as single package formulations with no mixing required, but with a shorter shelf life. Ordinarily the dye-relaxer formulations are prepared in two packages, with mixing required before use, and with extended shelf life.

When a two package formulation is desired, the dye portion contains, by weight, 0.05-10% dye, 1-25% emulsifier, 1-15% benzyl alcohol, 0.01-1% preservative and 35-60% water.

In that case, the relaxer portion contains, by weight, 1-7% alkali, 0.5-3% conditioning agent, 7-25% emulsifier, 5-15% moisturizer, 10-32% emollient, and 35-55% water.

The dye and relaxer portions are mixed together immediately before use, generally in a ratio, by weight, of approximately 9 parts relaxer to 1 part dye.

A single package formulation will contain, by weight, 1-7% alkali, 0.1-10% dye, 5-15% moisturizer, 10-32% emollient, 7.5-25% emulsifier, 0.5-3% conditioner and 35-55% water.

When the alkali relaxer is calcium hydroxide, an additional package is necessary, the additional package containing an activator composition comprising 15-35% by weight guanidine carbonate and 62-85% by weight water. Upon mixing with the calcium hydroxide-containing formulation guanidine hydroxide is formed.

When the alkali is sodium hydroxide, the concentration should be kept under about 3% by weight and when the alkali is lithium hydroxide, the concentration should be kept under about 5% by weight. For calcium hydroxide, up to about 7% by weight may be used.

Lithium hydroxide is the preferred alkali for the compositions of the invention, as it effectively straightens hair at a lower pH than sodium hydroxide, and is therefore less damaging to the hair. Lithium hydroxide is preferably present in the compositions of the invention in amounts of less than about 4% by weight. Compositions containing no more than about 2.5% by weight lithium hydroxide are preferred.

### EXAMPLES

In the following examples, all percentages are by weight.

### Example 1

A two-part hair relaxing-dye composition was prepared in which the relaxer formulation had the following composition:

| | |
|---|---|
| Cetearyl Alcohol | 8.00% |
| Ceteareth-20 | 1.85% |
| Cetyl Alcohol | 1.62% |
| PEG-75 Lanolin | 0.98% |
| Mineral Oil | 23.64% |
| Petrolatum | 9.86% |
| DEA-Oleth-10 Phosphate | 0.35% |
| Deionized Water | 46.35% |
| Propylene Glycol | 2.95% |
| Lithium Hydroxide | 2.52% |
| Polymethacrylamidopropyl- | 1.47% |
| trimonium Chloride | |
| Fragrance | 0.40% |

A black dye solution for mixing with the above relaxer formulation had the following composition:

| | |
|---|---|
| Basic Blue 99 (Arianor ® Steel Blue) | 5.00% |
| Basic Red 76 (Arianor ® Madder Red) | 1.5% |
| PEG-4 | 25.00% |
| Benzyl Alcohol | 10.00% |
| DMDM Hydantoin | 0.20% |
| Methylparaben | 0.10% |
| Deionized Water | 58.20 |

### Example 2

A single package hair relaxing-dye composition was prepared having the following composition:

| | |
|---|---|
| Cetearyl Alcohol | 8.00% |
| Cetyl Alcohol | 1.62% |
| Ceteareth-20 | 1.85% |
| PEG-4 | 2.50% |
| Mineral Oil | 23.64% |
| Petrolatum | 9.86% |
| DEA-Oleth-10 Phosphate | 0.35% |
| Deionized Water | 42.00% |
| Propylene Glycol | 2.95% |
| Lithium Hydroxide | 2.50% |
| Polymethacrylamidopropyl | 1.62% |
| trimonium Chloride | |
| PEG-75 Lanolin | 0.98% |
| Benzyl Alcohol | 1.00% |
| Basic Blue 99 | 0.50% |
| (Arianor ® Steel Blue) | |
| Basic Red 76 | 0.18% |
| (Arianor ® Madder Red) | |
| Tocopherol | 0.01% |
| Fragrance | 0.40% |

### Example 3

A three-package hair relaxing system was prepared, in which the relaxer had the following composition:

| | |
|---|---|
| Cetearyl Alcohol | 8.70% |
| Cetyl Alcohol | 1.94% |
| Ceteareth-20 | 2.00% |
| PEG-75 Lanolin | 0.97% |
| Mineral Oil | 23.35% |
| Petrolatum | 4.80% |
| DEA-Oleth-10 Phosphate | 0.48% |
| Deionized Water | 48.06% |
| Propylene Glycol | 2.25% |
| Calcium Hydroxide | 6.00% |
| Polymethacrylamidopropyl | 1.45% |
| trimonium Chloride | |

An activator composition was prepared having the following composition:

| | |
|---|---|
| Deionized Water | 77.12% |
| Guanidine Carbonate | 22.78% |
| Xanthan Gum | 0.10% |
| D&C Red #40 | 0.0018% |

A black dye solution for mixing with the above relaxer and activator formulation had the following composition:

| | |
|---|---|
| Basic Blue 99 (Arianor ® Steel Blue) | 5.00% |
| Basic Red 76 (Arianor ® Madder Red) | 1.5% |
| PEG-4 | 25.00% |
| Benzyl Alcohol | 10.00% |
| DMDM Hydantoin | 0.20% |
| Methylparaben | 0.10% |
| Deionized Water | 58.20 |

### Example 4

A two-part hair relaxing-dye composition containing sodium hydroxide was prepared in which the relaxer formulation had the following composition:

| | |
|---|---|
| Cetearyl Alcohol | 10.00% |
| Cetyl Alcohol | 1.00% |
| PEG-75 Lanolin | 0.50% |
| Mineral Oil | 13.95% |
| Petrolatum | 22.91% |
| DEA-Oleth-10 Phosphate | 0.50% |
| Deionized Water | 43.60% |
| Propylene Glycol | 5.00% |
| Sodium Hydroxide | 2.16% |
| Polymethacrylamidopropyl- | 1.50% |
| trimonium Chloride | |

A black dye solution for mixing with the above relaxer formulation had the following composition:

| | |
|---|---|
| Basic Blue 99 (Arianor ® Steel Blue) | 5.00% |
| Basic Red 76 (Arianor ® Madder Red) | 1.5% |
| PEG-4 | 25.00% |
| Benzyl Alcohol | 10.00% |
| DMDM Hydantoin | 0.20% |
| Methylparaben | 0.10% |
| Deionized Water | 58.20 |

### Example 5

### Comparison Test

A hair relaxer-colorant was prepared according to instructions found in the Arianor® catalog, by pouring an Arianor® dispersion containing emulsifier, thickener and carrier into a sodium hydroxide solution immediately before application. The result of the mixing is a gel having the following composition, by weight:

| | |
|---|---|
| Deionized water | to 100% |
| Sodium Hydroxide | 2.00% |
| Liquid Paraffin BP | 20.00 (Carrier) |
| Crodalan AWS | 5.00 (Emulsifier) |
| Natrosol 250HHX | 3.00 (Thickener) |
| Arianor ® Colours | 1.00 (Colorant) |

The above composition was compared to a colorant prepared according to Example 1, above. Both compositions were applied to a test hair swatch. The results are set forth below:

### Arianor®-suggested Composition

Shade: Composition has a deeper, darker, shade before application, and left a light, dull shade on hair swatch. Application: Easily spread.

Consistency: Gel

Relaxation: Adequate/good

Condition/Feel: Left hair feeling rough and dry.

Rinseability: Easily rinsed.

Combability: Normal to difficult.

pH: 13.5 (high alkali) High pH causes over-swelling of the cuticle and is more likely to cause damage, specifically in the form of breakage, to the hair.

### Example 1 Composition

Shade: Composition has a lighter, brighter shade before application and left a richer, brighter shade on hair swatch.

Application: Easily spread.

Consistency: Cream

Relaxation: Very good

Condition/Feel: Leaves hair feeling soft and smooth.

Rinseability: Easily rinsed.

Combability: Good

pH: 11.5 (alkali)

The lower pH causes sufficient swelling for color and conditioner to be locked into cuticle, while minimizing damage to the hair.

What is claimed is:

1. A composition for straightening and relaxing hair, comprising, by weight, about 0.1-10% of at least one cationic dye, about 1-7% of an alkali relaxer, about 7-25% of an emulsifier, about 10-32% of an emollient, about 5-15% of a moisturizer, about 0.5-3% of a conditioning agent and about 35-55% of water.

2. The composition of claim 1, wherein the conditioning agent is polymethacrylamidopropyltrimonium chloride.

3. The composition of claim 1, wherein the emulsifier is selected from the group consisting of cetyl alcohol, cetearyl alcohol, ceteareth-20, DEA-oleth-10 phosphate and mixtures thereof.

4. The composition of claim 1, wherein the alkali is selected from the group consisting of sodium hydroxide, lithium hydroxide and calcium hydroxide and mixtures thereof.

5. The composition of claim 4, wherein the alkali comprises calcium hydroxide, and the composition additionally comprises guanidine hydroxide as an activator.

6. The composition of claim 1, wherein the emollient is selected from the group consisting of mineral oil, PEG-75 lanolin and mixtures thereof.

7. The composition of claim 1, wherein the moisturizer comprises petrolatum.

8. The composition of claim 1, wherein the cationic dye is selected from the group consisting of:
(W—NH—Z−X⁺) Y⁻,
(W—N═N—Z−X⁺)Y⁻
and mixtures thereof,
where Z is an optionally substituted single benzene ring or a naphthalene ring, W is an optionally substituted ring with 5 or 6 members, or a naphthalene ring, X⁺ is a quaternary ammonium group of formula (R₁R₂R₃)N⁺ where R₁, R₂, and R₃ are, independently, short chain alkyl groups with 1-4 carbons, and Y⁻ is an anion.

9. The composition of claim 8, wherein the benzene ring is substituted with at least one group selected from the group consisting of —NO₂, —NH₂, ═NH, —OCH₃, ═O and Br.

10. The composition of claim 8, wherein Z is substituted with at least one group selected from the group consisting of —OH and —CH₃.

11. The composition of claim 8, wherein X⁺ is trimethyl ammonium.

12. The composition of claim 8, containing at least one dye selected from the group consisting of:

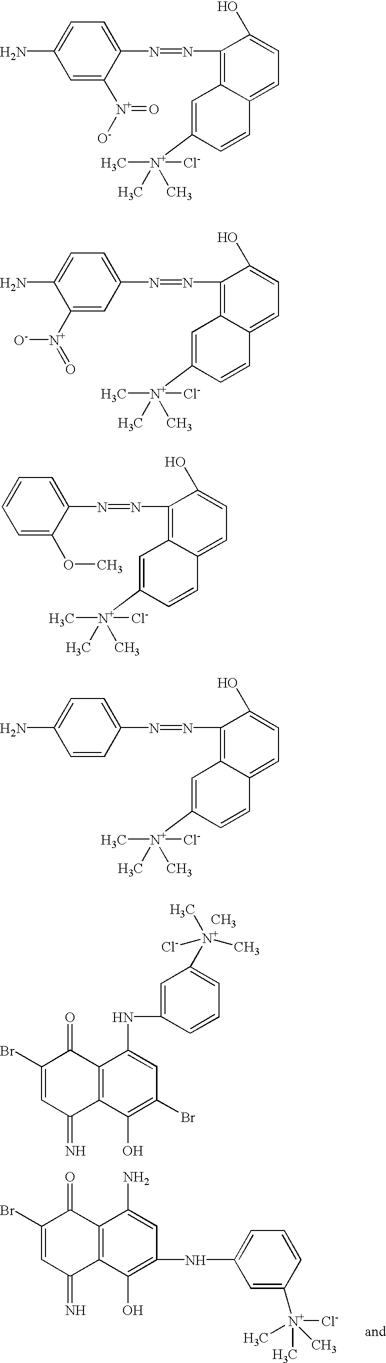

13. The composition of claim 1, which is a black dye containing a blue dye and a red dye in a blue to red weight ratio of about 3:1 to 4:1.

14. The composition of claim 1, which is contained in two packages for mixing before use,
wherein a first package contains a relaxer formulation comprising, by weight, about 1-7% of an alkali relaxer, about 7-25% of an emulsifier, about 10-32% of an emollient, about 5-15% of a moisturizer, about 0.5-3% of a conditioning agent and about 35-55% of water, and
a second package contains a dye formulation comprising, by weight, about 0.1-10% of at least one cationic dye, about 1-25% of an emulsifier, and about 35-60% of water.

15. The composition of claim 14, wherein the alkali relaxer comprises calcium hydroxide, and the composition additionally comprise a third package containing guanidine carbonate and water.

16. The composition of claim 4, wherein the alkali is lithium hydroxide in an amount of less than about 5% by weight.

17. The composition of claim 16, wherein the alkali is lithium hydroxide in an amount of less than about 4% by weight.

18. The composition of claim 17, wherein the alkali is lithium hydroxide in an amount of less than about 2.5% by weight.
